(19) United States
(12) Patent Application Publication
(10) Pub. No.: US 2022/0296172 A1
(43) Pub. Date: Sep. 22, 2022

(54) METHOD AND SYSTEM FOR ESTIMATING ARTERIAL BLOOD BASED ON DEEP LEARNING

(71) Applicant: Electronics and Telecommunication Research Institute, Daejeon (KR)

(72) Inventor: Yong Sik JIN, Daejeon (KR)

(21) Appl. No.: 17/584,246

(22) Filed: Jan. 25, 2022

(30) Foreign Application Priority Data
Mar. 16, 2021 (KR) 10-2021-0033974

(51) Int. Cl. A61B 5/00; A61B 5/024; A61B 5/021; G16H 50/20

(52) U.S. Cl. CPC A61B 5/7267; A61B 5/02416; A61B 5/02108; A61B 5/7278; A61B 5/7203; G16H 50/20

(57) ABSTRACT

Provided is a system for estimating arterial blood pressure based on deep learning. The system includes a sensing device configured to sense photoplethysmography from a predetermined body part and an arterial blood pressure estimation device which trains the learning model by setting the photoplethysmography provided from the sensing device as an input variable of a predetermined learning model that is pre-constructed and setting an estimated target value of the arterial blood pressure in a current time section and a predicted target value of the arterial blood pressure after the current time section as an output variable and which outputs the estimated target value or the predicted target value of the arterial blood pressure by performing deep learning on the photoplethysmography based on the learning model.

### CROSS-REFERENCE TO RELATED APPLICATION

This application claims priority to and the benefit of Korean Patent Application No. 10-2021-0033974, filed on Mar. 16, 2021, the disclosure of which is incorporated herein by reference in its entirety.

### BACKGROUND

1. Field of the Invention

The present invention relates to a method and system for estimating arterial blood pressure based on deep learning and, more particularly, to a method and system for estimating a current arterial blood pressure value in a non-invasive method based on deep learning and predicting a future arterial blood pressure value to predict whether blood pressure is abnormal.

2. Discussion of Related Art

Blood pressure is one of the important biological signals for determining a person's health condition. Blood pressure that is much higher than a blood pressure of a normal person is called hypertension, and blood pressure that is much lower than blood pressure of a normal person is called hypotension. A treatment standard for pathological hypertension is diastolic blood pressure, and when the diastolic blood pressure is 90 mmHg or higher, it is generally a target to be treated. When the hypertension is left untreated, there is a risk of fatal diseases such as cerebral hemorrhage. As such, blood pressure may be an index for determining whether to treat hypertensive or hypotensive patients.

Currently, various blood pressure measuring devices based on an invasive or non-invasive method are being researched and developed. However, in the case of a blood pressure measuring device based on the non-invasive method, it is difficult to observe continuous changes in blood pressure, such as calculating only systolic blood pressure or diastolic blood pressure.

[Related Art Document]

[Patent Document]

Korean Patent Laid-Open Publication No. 10-2016-0108081(Sep. 19, 2016)

### SUMMARY OF THE INVENTION

The present invention is directed to providing a method and system for estimating arterial blood pressure based on deep learning that are capable of calculating an estimated target value of continuous arterial blood pressure in a current time section through deep learning after sensing photoplethysmography, and providing a future predicted target value of arterial blood pressure to easily check and predict whether blood pressure is abnormal.

However, the problems to be solved by the present invention are not limited to the problems described above, and other problems may be present.

According to an aspect of the present invention, there is provided a method of estimating arterial blood pressure based on deep learning, including: sensing photoplethysmography from a predetermined body part; training a predetermined learning model that is pre-constructed by setting the sensed photoplethysmography as an input variable and setting an estimated target value of the arterial blood pressure in a current time section and a predicted target value of the arterial blood pressure after the current time section as an output variable; and outputting the estimated target value or the predicted target value of the arterial blood pressure by inputting the photoplethysmography based on the learning model and then performing the deep learning.

According to another aspect of the present invention, there is provided a system for estimating arterial blood pressure based on deep learning, including: a sensing device configured to sense photoplethysmography from a predetermined body part; and an arterial blood pressure estimation device which trains the learning model by setting the photoplethysmography provided from the sensing device as an input variable of a predetermined learning model that is pre-constructed and setting an estimated target value of the arterial blood pressure in a current time section and a predicted target value of the arterial blood pressure after the current time section as an output variable and which outputs the estimated target value or the predicted target value of the arterial blood pressure by performing deep learning on the photoplethysmography based on the learning model.

A computer program according to another aspect of the present invention for solving the above-described problems is combined with a computer that is hardware to execute the method of estimating arterial blood pressure based on deep learning and is stored in a computer-readable recording medium.

Other specific details of the invention are contained in the detailed description and drawings.

The above-described configurations and operations of the present invention will become more apparent from embodiments described in detail below with reference to the accompanying drawings.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of a system for estimating arterial blood pressure based on deep learning according to an embodiment of the present invention;

FIG. 2A is a diagram for describing content of outputting an estimated target value of the arterial blood pressure based on photoplethysmography;

FIG. 2B is a diagram for describing contents of outputting estimated values of a relaxation value and a contraction value of the arterial blood pressure;

FIG. 3A is a diagram for describing contents of outputting an estimated target value of the arterial blood pressure based on photoplethysmography;

FIG. 3B is a diagram for describing contents of outputting predicted target values of a diastolic blood pressure value and a systolic blood pressure value of the arterial blood pressure;

FIG. 4A is a diagram for describing contents of outputting the predicted target value of the arterial blood pressure based on the photoplethysmography and the estimated target value of the arterial blood pressure;

FIG. 4B is a diagram for describing contents of outputting predicted values of the relaxation value and the contraction value of the arterial blood pressure based on the photoplethysmography and the estimated value of the arterial blood pressure;

FIGS. 5A and 5B are diagrams for describing prediction of an abnormal symptom of an arterial blood pressure according to a first embodiment;

FIG. 5C is a diagram for describing prediction of an abnormal symptom of an arterial blood pressure according to a second embodiment;

FIG. 5D is a diagram for describing prediction of an abnormal symptom of an arterial blood pressure according to a third embodiment;

FIG. 6 is a block diagram illustrating an apparatus for estimating an arterial blood pressure according to an embodiment of the present invention; and

FIG. 7 is a flowchart of a method of estimating arterial blood pressure based on deep learning according to an embodiment of the present invention.

### DETAILED DESCRIPTION OF EXEMPLARY EMBODIMENTS

Various advantages and features of the present invention and methods accomplishing them will become apparent from the following description of embodiments with reference to the accompanying drawings. However, the present invention is not limited to the embodiments disclosed herein but will be implemented in various forms. The embodiments serve to make the present invention thorough and are provided so that those skilled in the art can easily understand the scope of the present invention. Therefore, the present invention will be defined by the scope of the appended claims.

Terms used in the present specification are for explaining the embodiments rather than limiting the present invention. In the present invention, a singular form includes a plural form unless explicitly described to the contrary. Throughout this specification, the term “comprise” and/or “comprising” will be understood to imply the inclusion of stated constituents but not the exclusion of any other constituents. Like reference numerals refer to like elements throughout the specification and “and/ or” includes each of the above-described components and includes all combinations thereof. Although “first,” “second,” and the like are used to describe various components, it goes without saying that these components are not limited by these terms. These terms are used only to distinguish one component from other components. Therefore, it goes without saying that the first component described below may be the second component within the technical scope of the present invention.

Unless otherwise defined, all terms (including technical and scientific terms) used in the present specification have the same meaning as meanings commonly understood by those skilled in the art to which the present invention pertains. In addition, terms as defined in commonly used dictionary are not to be ideally or excessively interpreted unless explicitly defined otherwise.

The present invention relates to a method and system 100 capable of estimating a current arterial blood pressure value and predicting a future arterial blood pressure value using a non-invasive method based on deep learning to predict whether blood pressure is abnormal.

Blood pressure refers to pressure applied to a blood vessel as blood flows. As a method of measuring such blood pressure, there are an invasive method and a non-invasive method.

First, the invasive method has the advantage of continuously and accurately measuring blood pressure but has a technical problem of inserting a catheter into a blood vessel and a problem of causing infection and side effects accordingly.

Next, as the non-invasive method, an auscultatory method of measuring blood pressure using a Korotkoff sound is the most representative blood pressure measurement method. In addition, there is an oscillometry method of measuring blood pressure using vibration generated by a flow of blood. However, there is a disadvantage in that the non-invasive blood pressure measurement method requires cuff inflation and contraction, requires a measurement interval of at least 30 minutes, is inconvenient to attach and carry, and it is impossible to continuously measure blood pressure. In addition, among the non-invasive methods, a tonometer has an advantage of providing a continuous blood pressure waveform but has a disadvantage in that it was very sensitive to a movement of a wrist and a position of a sensor.

In order to compensate for the shortcomings of these non-invasive blood pressure measurement methods, a method of non-invasively and continuously measuring blood pressure using a pulse transit time (PTT) using a delay time between an R wave of a recent electrocardiogram (ECG) and a peak of photoplethysmography (PPG) is being studied.

Since these methods are designed to calculate only systolic blood pressure (SBP) or diastolic blood pressure (DBP), there is a problem in that it is difficult to observe a continuous change in blood pressure.

In order to improve these problems, an embodiment of the present invention calculates an estimated target value of arterial blood pressure in a current time section through deep learning after sensing PPG and provides a future predicted target value of the arterial blood pressure, thereby making it possible to easily check and predict an abnormal state of blood pressure.

Hereinafter, a system for estimating arterial blood pressure based on deep learning according to an embodiment of the present invention will be described with reference to the accompanying drawings.

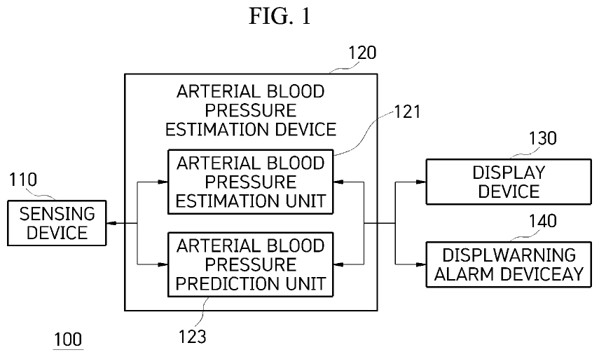

FIG. 1 is a block diagram of a system 100 for estimating arterial blood pressure based on deep learning according to the embodiment of the present invention.

The system 100 for estimating arterial blood pressure according to the embodiment of the present invention includes a sensing device 110 and an arterial blood pressure estimation device 120.

First, the sensing device 110 senses PPG from sensors attached to a predetermined body part.

In an embodiment, the sensing device 110 for measuring the PPG outputs a pulse wave as a change in electrical resistance using a photoconductive element such as a CdS cell. A series of processes of measuring the PPG by attaching the sensing device 110 at a predetermined position of a human body, such as a finger, an ear, or a wrist, can be understood by a person skilled in the art related to the embodiment of the present invention.

Next, the arterial blood pressure estimation device 120 includes an arterial blood pressure estimation unit 121 and an arterial blood pressure prediction unit 123.

In this case, the arterial blood pressure estimation unit 121 and the arterial blood pressure prediction unit 123 use a predetermined learning model that is pre- constructed. In this case, as the learning model used in the embodiment of the present invention, a deep learning model may be applied. For example, deep learning techniques, such as a fully convolutional network (FCN), a convolutional neural network (CNN), a generative adversarial network (GAN), a recurrent neural network (RNN), an autoencoder (AE), and a neural ODE, may be used.

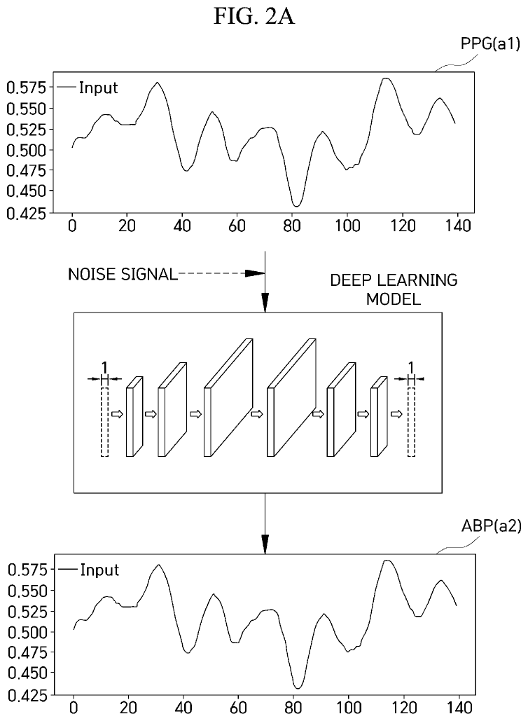

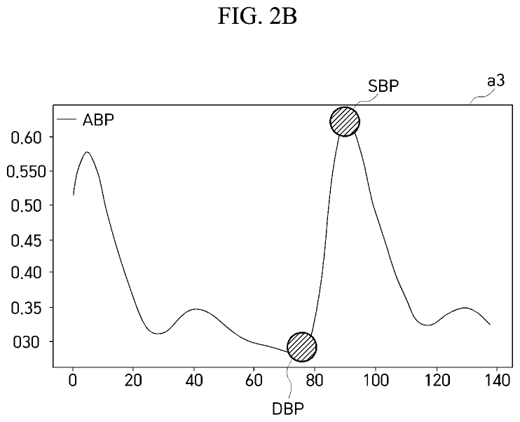

FIG. 2A is a diagram for describing contents of outputting an estimated target value of the arterial blood pressure based on the PPG. FIG. 2B is a diagram for describing contents of outputting estimated target values of a diastolic blood pressure value and a systolic blood pressure value of the arterial blood pressure.

The arterial blood pressure estimation unit 121 sets PPG (a1) sensed through the measuring device as an input variable, and sets an estimated target value (a2) of the arterial blood pressure in the current time section as an output variable to train the pre-constructed learning model.

In this case, in an embodiment of the present invention, in order to be robust to noise during training of the learning model, noise may be generated virtually and applied together with the input variable, or the actually generated and measured noise may be applied together with the input variable. Such a noise signal may be additionally used only during the training of the learning model. That is, during the training, the noise signal is added to an input signal to perform the training, and for subsequent estimation or prediction, an estimated target value or a predicted target value is obtained using only the input signal.

As the training process of the learning model is completed, the arterial blood pressure estimation unit 121 inputs the PPG (a1) based on the learning model and then performs the deep learning to output the estimated target value (a2) of the arterial blood pressure corresponding to the current time section.

In addition, the arterial blood pressure estimation unit 121 may calculate estimated target values (a3) of the DBP value and the SBP value of the arterial blood pressure, respectively, from a maximum value and a minimum value of the estimated target value (a2) of the arterial blood pressure.

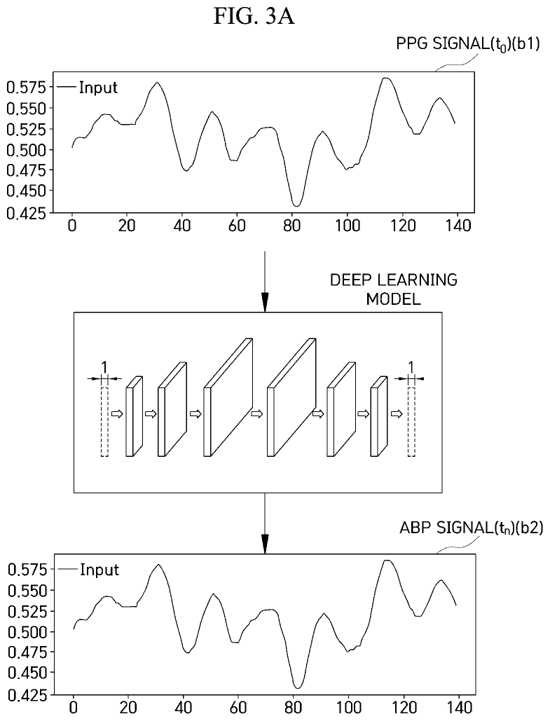

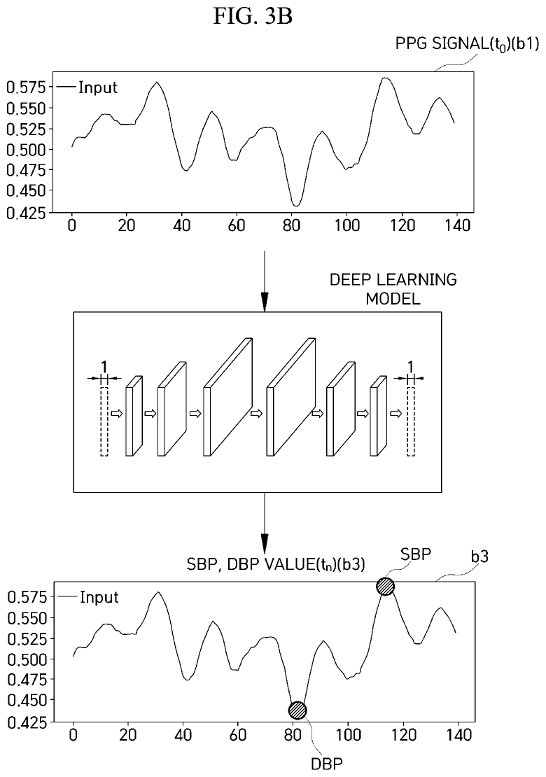

FIG. 3A is a diagram for describing contents of outputting the predicted target value of the arterial blood pressure based on the PPG. FIG. 3B is a diagram for describing contents of outputting predicted target values of the DBP value and the SBP value of the arterial blood pressure. In this case, to illustrated in the drawing represents the current time section, and to represents time data after the current time section.

Next, the arterial blood pressure prediction unit 123 sets only sensed PPG (b1) as an input variable, and sets a predicted target value (b2) of the arterial blood pressure after the current time section as an output variable to train a predetermined learning model.

Similarly, in the embodiment of the present invention, in order to be robust to noise during the training of the learning model, noise may be generated virtually and applied together with the input variable, or the actually generated and measured noise may be applied together with the input variable.

The arterial blood pressure prediction unit 123 inputs the PPG (b1) based on the trained learning model through the training process and then performs the deep learning to output the corresponding predicted target value (b2) of the arterial blood pressure after the current time section.

In addition, the arterial blood pressure estimation unit 121 may calculate estimated target values (a3) of the DBP value and the SBP value of the arterial blood pressure, respectively, from a maximum value and a minimum value of the predicted target value (b1) of the arterial blood pressure.

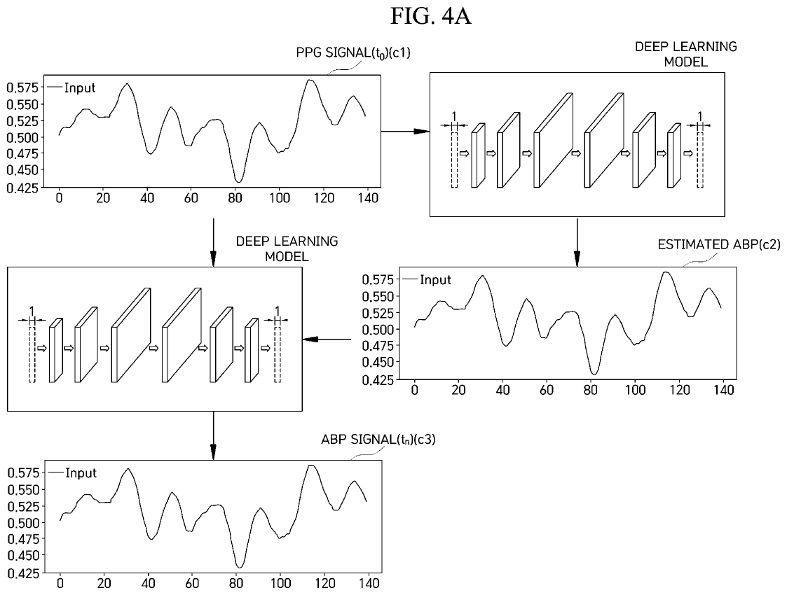

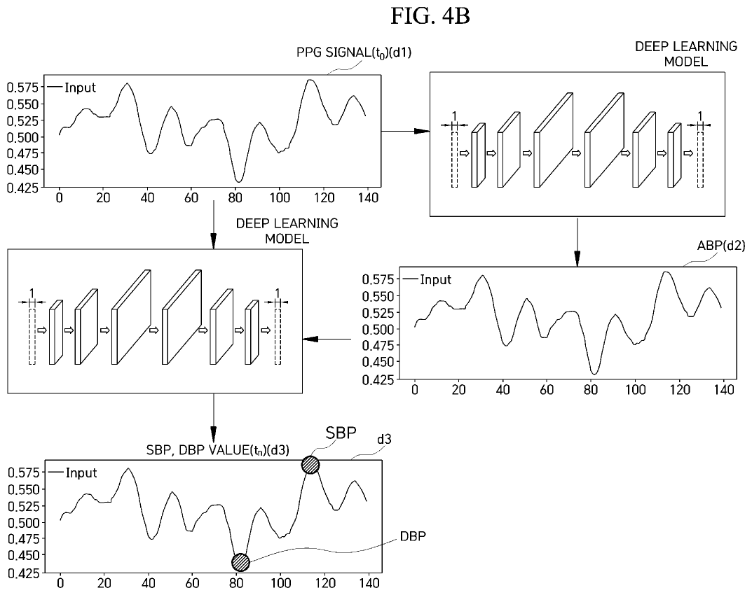

FIG. 4A is a diagram for describing contents of outputting the predicted target value of the arterial blood pressure based on the estimated target value of the arterial blood pressure. FIG. 4B is a diagram for describing contents of outputting the DBP value of the arterial blood pressure and the predicted target value of the SBP value based on the PPG and the estimated target values of the arterial blood pressure.

In another embodiment, the arterial blood pressure prediction unit 123 sets an estimated target value (c2) of the arterial blood pressure by the arterial blood pressure estimation unit 121 together with PPG (c1) in the current time section as the input variable of the learning model and sets a predicted target value (c3) of the arterial blood pressure after the current time section as the output variable to train the learning model.

Thereafter, the arterial blood pressure prediction unit 123 inputs the PPG (c1) and the estimated target value (c2) of the arterial blood pressure based on the trained learning model and then performs the deep learning to predict the predicted target value (c3) of the arterial blood pressure.

Similarly, the arterial blood pressure prediction unit 123 may input PPG (d1) and an estimated target value (d2) of the arterial blood pressure and then perform the deep learning to calculate predicted target values (d3) of the DBP value and the SPB value of the arterial blood pressure from a maximum value and a minimum value of the predicted target value (d2) of the arterial blood pressure.

In still another embodiment, the arterial blood pressure prediction unit 123 may set an actual measurement value of the arterial blood pressure instead of the estimated target value of the arterial blood pressure, together with the PPG in the current time section as the input variable of the learning model, and set the predicted target value of the arterial blood pressure after the current time section as the output variable to train the learning model.

Thereafter, the arterial blood pressure prediction unit 123 may input the PPG and the measurement value of the arterial blood pressure based on the trained learning model and then perform the deep learning to output the predicted target value of the arterial blood pressure.

When the estimated target value or the predicted target value of the arterial blood pressure is calculated through this process, the arterial blood pressure estimation unit 121 and the arterial blood pressure prediction unit 123 store the input data and the estimated target value or the predicted target value, and display the estimated target value or the predicted target value of the arterial blood pressure through the display device 130.

In addition, when the estimated target value or the predicted target value of the arterial blood pressure through the warning alarm device 140 deviates from the preset threshold range, that is, when the blood pressure provided as the current estimated target value or the future blood pressure increases or decreases, a danger alarm may be provided to a user. In this case, the warning alarm device 140 may provide a danger alarm to a user through at least one of text, vibration, haptic, sound, and the display device 130. Meanwhile, a threshold range in the embodiment of the present invention may be set as a threshold value which is constant or set based on a difference between the current estimated target value and the predicted target value.

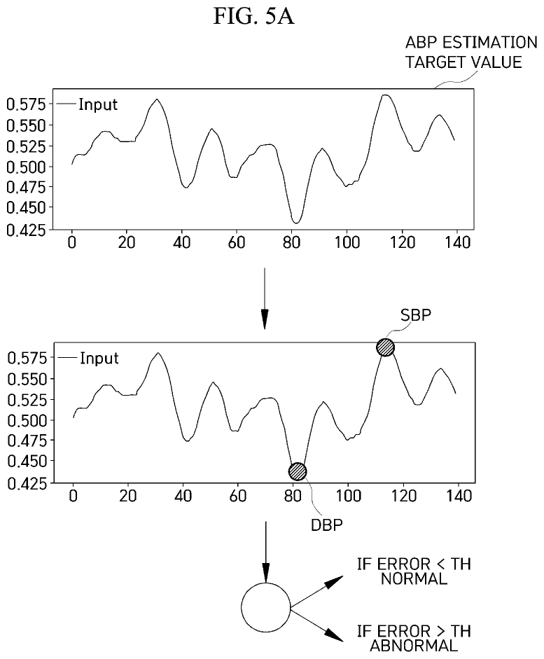

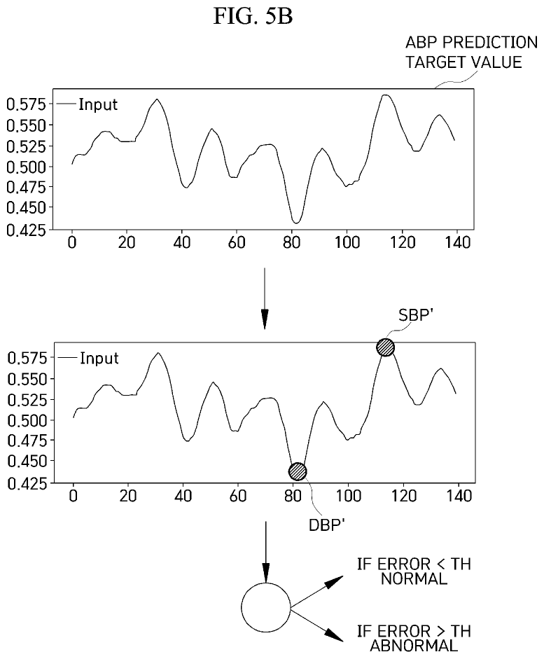

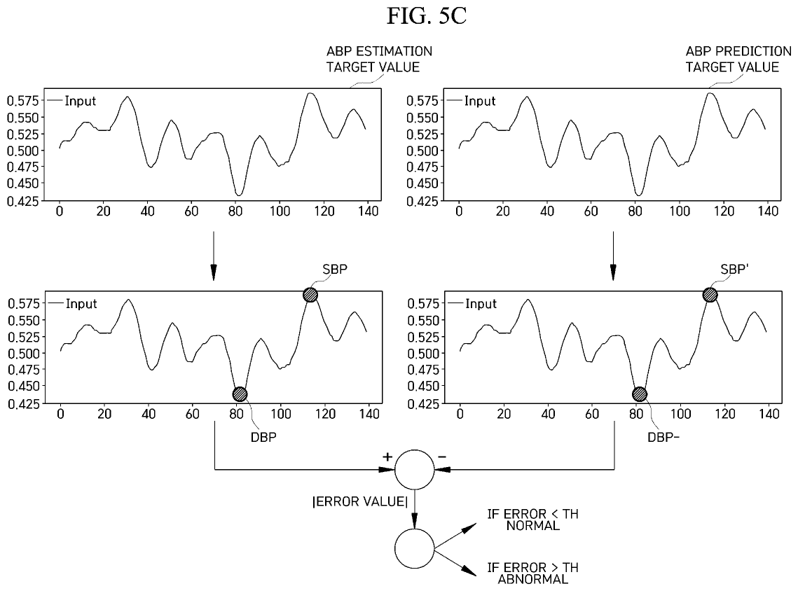

FIGS. 5A and 5B are diagrams for describing prediction of an abnormal symptom of an arterial blood pressure according to a first embodiment.

An arterial blood pressure estimation device 120 according to the embodiment of the present invention may determine that there is an abnormal symptom of arterial blood pressure and provide a danger alarm through a warning alarm device 140 when at least one of diastolic and systolic blood pressure values of the arterial blood pressure calculated from an estimated target value of the arterial blood pressure corresponding to the current time section is greater than each preset threshold value.

Similarly, the arterial blood pressure estimation device 120 may determine that the abnormal symptom of the arterial blood pressure is present and provide the danger alarm through the warning alarm device 140 when at least one of the diastolic and systolic blood pressure values of the arterial blood pressure calculated from the predicted target value after the current time section is greater than each preset threshold value (see FIG. 5B).

FIG. 5C is a diagram for describing prediction of an abnormal symptom of an arterial blood pressure according to a second embodiment.

In addition, an arterial blood pressure estimation device 120 according to the embodiment of the present invention may calculate an error value between diastolic and systolic blood pressure values of an arterial blood pressure calculated from an estimated target value of the arterial blood pressure corresponding to a current time section and a predicted target value after the current time section, respectively, and determine that there is an abnormal symptom of the arterial blood pressure and provide a danger alarm through a warning alarm device 140 when the error value is greater than a preset threshold value.

FIG. 5D is a diagram for describing prediction of an abnormal symptom of an arterial blood pressure according to a third embodiment.

In addition, an arterial blood pressure estimation device 120 according to the embodiment of the present invention may use an error signal of an estimated target value (e1) and a predicted target value (e2) of an arterial blood pressure to predict an abnormal symptom of the arterial blood pressure.

That is, the arterial blood pressure estimation device 120 may compare the calculated error signal with a preset threshold value and predict the abnormal symptom of the arterial blood pressure based on the comparison result.

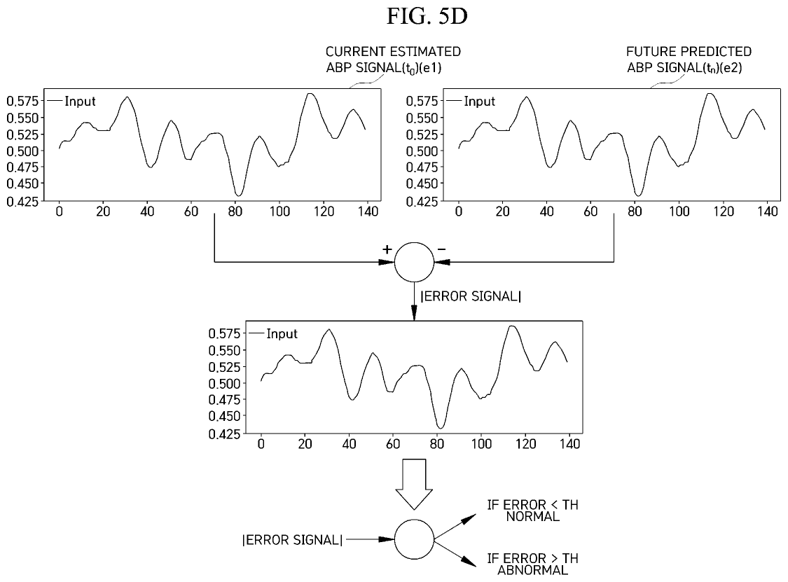

FIG. 6 is a block diagram of the arterial blood pressure estimation device 120 according to the embodiment of the present invention.

The arterial blood pressure estimation device 120 according to the above description may include a communication module 10, a memory 20, and a processor 30.

The communication module 10 receives the PPG sensed by the sensing device 110.

The memory 20 stores a program for training the learning model based on the PPG and outputting the estimated target value or the predicted target value of the arterial blood pressure based on the trained learning model, and the processor 30 executes the program stored in the memory 20.

For reference, the components illustrated in FIG. 1 according to the embodiment of the present invention may be implemented in the form of software or hardware, such as a field programmable gate array (FPGA) or application specific integrated circuit (ASIC), and perform predetermined roles.

However, “components” are not limited to software or hardware, and each component may be configured to be in an addressable storage medium or to reproduce one or more processors.

Accordingly, for example, a component includes components such as software components, object-oriented software components, class components, and task components, processors, functions, attributes, procedures, subroutines, segments of a program code, drivers, firmware, a microcode, a circuit, data, a database, data structures, tables, arrays, and variables.

Components and functions provided within the components may be combined into a smaller number of components or further divided into additional components.

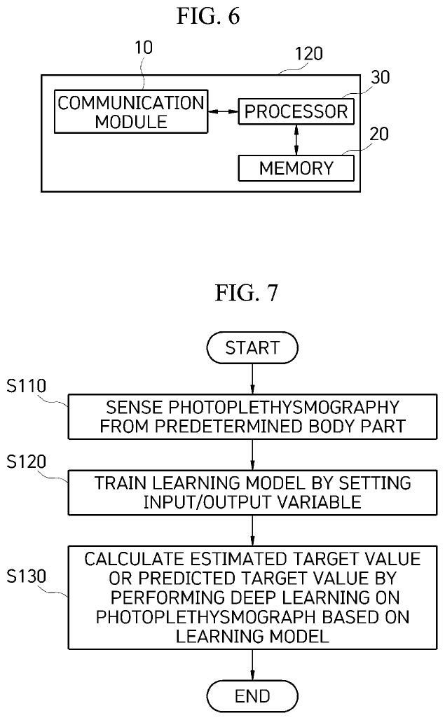

Hereinafter, a method of estimating arterial blood pressure based on deep learning according to an embodiment of the present invention will be described with reference to FIG. 7.

FIG. 7 is a flowchart of a method of estimating arterial blood pressure based on deep learning according to an embodiment of the present invention.

Meanwhile, each operation illustrated in FIG. 7 may be understood to be performed by a server or a device constituting the system 100 for estimating arterial blood pressure based on deep learning but is not necessarily limited thereto.

First, the PPG is sensed from sensors attached to a predetermined body part (S110).

Next, the predetermined learning model that is pre-constructed by setting the sensed PPG as the input variable and setting the estimated target value of the arterial blood pressure in the current time section and the predicted target value of the arterial blood pressure after the current time section as the output variable is trained (S120).

Next, the estimated target value or the predicted target value of the arterial blood pressure is output by inputting the PPG based on the learning model and then performing the deep learning (S130).

Meanwhile, in the above description, in operations S110 to S130, additional steps may be further added or divided or may be combined into fewer steps according to an implementation example of the present invention. Also, some steps may be omitted if necessary, and an order between the operations may be changed. In addition, even if other contents are omitted, the contents of FIGS. 1 to 6 are also applied to the method of estimating arterial blood pressure based on deep learning of FIG. 7.

The above-described embodiment of the present invention may be embodied as a program (or application) and stored in a medium for execution in combination with a computer which is hardware.

In order for the computer to read the program and execute the methods implemented as the program, the program may include a code coded in a computer language such as C, C++, JAVA, Ruby, or machine language that the processor (CPU) of the computer may read through a device interface of the computer. Such code may include functional code related to a function or such defining functions necessary for executing the methods and may include an execution procedure related control code necessary for the processor of the computer to execute the functions according to a predetermined procedure. In addition, the code may further include a memory reference related code for which location (address street number) in an internal or external memory of the computer the additional information or media necessary for the processor of the computer to execute the functions is to be referenced at. In addition, when the processor of the computer needs to communicate with any other computers, servers, or the like located remotely in order to execute the above functions, the code may further include a communication-related code for how to communicate with any other computers, servers, or the like using the communication module of the computer, what information or media to transmit/receive during communication, and the like.

The storage medium is not a medium that stores images therein for a while, such as a register, a cache, a memory, or the like, but means a medium that semi- permanently stores the images therein and is readable by an apparatus. Specifically, examples of the storage medium include, but are not limited to, a read-only memory (ROM), random-access memory (RAM), compact disc read-only memory (CD- ROM), a magnetic tape, a floppy disk, an optical image storage device, and the like. That is, the program may be stored in various recording media on various servers accessible by the computer or in various recording media on the computer of the user. In addition, media may be distributed in a computer system connected by a network, and a computer-readable code may be stored in a distributed manner.

The above description of the present invention is for illustrative purposes, and those skilled in the art to which the present invention pertains will understand that it is possible to be easily modified to other specific forms without changing the technical spirit or essential features of the present invention. Therefore, it is to be understood that the embodiments described hereinabove are illustrative rather than being restrictive in all aspects. For example, each component described as a single type may be implemented in a distributed manner, and similarly, components described as distributed may be implemented in a combined form.

According to the above-described embodiment of the present invention, it is possible to know an estimated target value of continuous arterial blood pressure through a non-invasive method and, furthermore, to check predicted target values of a diastolic blood pressure value and a systolic blood pressure value of the arterial blood pressure in advance.

Effects of the present invention are not limited to the above-described effects, and other effects that are not described will be clearly understood by those skilled in the art from the following descriptions.

It is to be understood that the scope of the present invention will be defined by the claims rather than the above-described description and all modifications and alternations derived from the claims and their equivalents are included in the scope of the present invention.

What is claimed is:

1. A method of estimating arterial blood pressure based on deep learning executed by a computer, the method comprising:
sensing photoplethysmography from a predetermined body part;
training a predetermined learning model that is pre-constructed by setting the sensed photoplethysmography as an input variable and setting an estimated target value of the arterial blood pressure in a current time section and a predicted target value of the arterial blood pressure after the current time section as an output variable; and
outputting the estimated target value or the predicted target value of the arterial blood pressure by inputting the photoplethysmography based on the learning model and then performing the deep learning.

2. The method of claim 1, wherein the learning model is pre-trained by including virtually generated noise or actually generated and measured noise.

3. The method of claim 1, wherein the outputting of the estimated target value or the predicted target value of the arterial blood pressure by inputting the photoplethysmography based on the learning model and then performing the deep learning includes calculating estimated target values or predicted target values of a diastolic blood pressure value and a systolic blood pressure value of the arterial blood pressure from a maximum value and a minimum value of the estimated target value of the arterial blood pressure or the predicted target value of the arterial blood pressure.

4. The method of claim 1, wherein the training of the learning model by setting the sensed photoplethysmography as the input variable and setting the estimated target value of the arterial blood pressure in the current time section and the predicted target value of the arterial blood pressure after the current time section as the output variable includes training the learning model by setting the photoplethysmography and the estimated target value of the arterial blood pressure in the current time section as the input variables of the learning model and setting the predicted target value of the arterial blood pressure after the current time section as the output variable, and
the outputting of the estimated target value or the predicted target value of the arterial blood pressure by inputting the photoplethysmography based on the learning model and then performing the deep learning includes outputting the predicted target value of the arterial blood pressure by inputting the photoplethysmography and the estimated target value of the arterial blood pressure based on the learning model and then performing the deep learning.

5. The method of claim 4, wherein the outputting of the estimated target value or the predicted target value of the arterial blood pressure by inputting the photoplethysmography based on the learning model and then performing the deep learning includes calculating predicted target values of a diastolic blood pressure value and a systolic blood pressure value of the arterial blood pressure from a maximum value and a minimum value of the predicted target value of the arterial blood pressure.

6. The method of claim 1, wherein the training of the predetermined learning model that is pre-constructed by setting the sensed photoplethysmography as the input variable and setting the estimated target value of the arterial blood pressure in the current time section and the predicted target value of the arterial blood pressure after the current time section as the output variable includes training the learning model by setting the photoplethysmography and an actual measurement value of the arterial blood pressure in the current time section as the input variable of the learning model and setting the predicted target value of the arterial blood pressure after the current time section as the output variable, and
the outputting of the estimated target value or the predicted target value of the arterial blood pressure by inputting the photoplethysmography based on the learning model and then performing the deep learning includes outputting the predicted target value of the arterial blood pressure by inputting the photoplethysmography and the measurement value of the arterial blood pressure based on the learning model and then performing the deep learning.

7. The method of claim 1, further comprising displaying the estimated target value or the predicted target value of the arterial blood pressure.

8. The method of claim 7, further comprising providing a warning alarm when the estimated target value or the predicted target value of the arterial blood pressure deviates from a preset threshold range.

9. The method of claim 1, further comprising:
calculating an error signal of the estimated target value and the predicted target value of the arterial blood pressure;
comparing the calculated error signal with a preset threshold value; and
predicting an abnormal symptom of the arterial blood pressure based on the comparison result.

10. The method of claim 1, further comprising:
calculating error values of diastolic and systolic blood pressure values for the estimated target value and the predicted target value of the arterial blood pressure;
comparing at least one of diastolic and systolic blood pressure values for the estimated target value and predicted target value of the arterial blood pressure, or the calculated error value with preset threshold values; and
predicting an abnormal symptom of the arterial blood pressure based on the comparison result.

11. A system for estimating arterial blood pressure based on deep learning, the system comprising:
a sensing device configured to sense photoplethysmography from a predetermined body part; and
an arterial blood pressure estimation device which trains a learning model by setting the photoplethysmography provided from the sensing device as an input variable of a predetermined learning model that is pre-constructed and setting an estimated target value of the arterial blood pressure in a current time section and a predicted target value of the arterial blood pressure after the current time section as an output variable and which outputs the estimated target value or the predicted target value of the arterial blood pressure by performing deep learning on the photoplethysmography based on the learning model.

12. The system of claim 11, wherein the arterial blood pressure estimation device trains the learning model by including virtually generated noise or actually generated and measured noise in training data.

13. The system of claim 11, wherein the arterial blood pressure estimation device calculates an estimated target value or a predicted target value of a diastolic blood pressure value and a systolic blood pressure value of the arterial blood pressure from a maximum value and a minimum value of the estimated target value of the arterial blood pressure or the predicted target value of the arterial blood pressure.

14. The system of claim 11, wherein the arterial blood pressure estimation device trains the learning model by setting the photoplethysmography and the estimated target value or an actual measurement value of the arterial blood pressure in the current time section as the input variable of the learning module in the current time section, and setting the predicted target value of the arterial blood pressure after the current time section as the output variable, and
the photoplethysmography and a measurement value of the arterial blood pressure or the actual measurement value of the arterial blood pressure are input based on the learning model, and the predicted target value of the arterial blood pressure is output.

15. The system of claim 14, wherein the arterial blood pressure estimation device calculates predicted target values of a diastolic blood pressure value and a systolic blood pressure value of the arterial blood pressure from a maximum value and a minimum value of the predicted target value of the arterial blood pressure.

16. The system of claim 11, further comprising a display configured to display the estimated target value or the predicted target value of the arterial blood pressure.

17. The system of claim 16, further comprising a warning alarm device configured to provide a warning alarm when the estimated target value or the predicted target value of the arterial blood pressure deviates from a preset threshold range.

18. The system of claim 11, wherein the arterial blood pressure estimating device calculates an error signal of the estimated target value and the predicted target value of the arterial blood pressure, compares the calculated error signal with a preset threshold value, and predicts an abnormal symptom of the arterial blood pressure based on the comparison result.

19. The system of claim 11, wherein the arterial blood pressure estimating device calculates an error value of diastolic and systolic blood pressure values for the estimated target value and the predicted target value of the arterial blood pressure, compares at least one of the diastolic and systolic blood pressure values for the estimated target value and the predicted target value of the arterial blood pressure or the calculated error value with preset threshold values, and predicts the abnormal symptom of the arterial blood pressure based on the comparison result.
